(19) United States
(12) Patent Application Publication
(10) Pub. No.: US 2004/0260271 A1
(43) Pub. Date: Dec. 23, 2004

(54) Extended fenestration catheter with internal coil and method of making the same

(76) Inventors: Richard F. Huyser, Kalamazoo, MI (US); Jason D. Toman, Portage, MI (US); Brian J. VanderWoude, Portage, MI (US); Steven J. Carusillo, Kalamazoo, MI (US)

Correspondence Address: HOWARD & HOWARD ATTORNEYS, P.C., THE PINEHURST OFFICE CENTER, SUITE #101, 39400 WOODWARD AVENUE, BLOOMFIELD HILLS, MI, 48304-5151, US

(21) Appl. No.: 10/870,887

(22) Filed: Jun. 17, 2004

Related U.S. Application Data
(60) Provisional application No. 60/479,612, filed on Jun. 18, 2003.

(51) Int. Cl. A61M 25/00

(52) U.S. Cl. 604/524

(57) ABSTRACT

A catheter for delivering medication from a fluid source to an anatomical site of a patient and a method of manufacturing the same are provided. The catheter comprises an elongated tube extending between an open proximal end and a closed distal end. The elongated tube includes a catheter wall having an inner surface that bounds a lumen for conveying the fluid from the fluid source. A plurality of openings are defined in the catheter wall to discharge the fluid from the lumen to the anatomical site. A reinforcing coil is disposed within the lumen to improve fluid delivery and provide kink resistance to the catheter. The reinforcing coil has spaced convolutions in an unstretched state. An annular flow space is defined between the reinforcing coil and the inner surface of the catheter wall. As a result, fluid can travel both through an inner lumen of the reinforcing coil and between the spaced convolutions to the annular flow space or the fluid can travel directly through the annular flow space to reach the openings.

### CROSS-REFERENCE TO RELATED APPLICATIONS

This application claims the benefit of U.S. provisional patent application Ser. No. 60/479,612, filed on Jun. 18, 2003, the advantages and disclosure of which are hereby incorporated by reference.

### FIELD OF THE INVENTION

The present invention relates generally to a catheter for providing uniform medication delivery to an anatomical site of a patient. More specifically, the present invention relates to the catheter having an internal coil to provide uniform medication delivery and kink resistance to the catheter.

### BACKGROUND OF THE INVENTION

A catheter conveys fluid such as medication from a fluid source to an anatomical site of a patient. A typical catheter comprises an elongated tube extending between an open proximal end and a closed distal end. The elongated tube includes a catheter wall having an inner surface with a lumen bound by the inner surface to convey the fluid from the fluid source. The elongated tube is often defined by two segments, a continuous segment, which is free of any holes or openings, and an infusion segment in which a plurality of openings are defined in the catheter wall. The plurality of openings discharges the fluid from the lumen into the anatomical site. In other words, each of the plurality of openings provides a corridor for the fluid from the lumen to enter the anatomical site.

When using the catheter for fluid delivery, air bubbles may be introduced into the lumen of the elongated tube due to connections and or valves used to connect the catheter to the fluid source. As the air moves toward the closed distal end of the elongated tube, the air obstructs fluid from exiting out of the plurality of openings in the infusion segment of the elongated tube. Ultimately, the air becomes permanently entrapped in the lumen in the infusion segment. The air either settles at the closed distal end of the elongated tube or gets trapped along the lumen in the proximity of the openings and permanently blocks fluid flow through all or a portion of the plurality of openings in the infusion segment. In certain instances, when the air becomes lodged in the infusion segment, only one or two of the most proximal openings are operative for fluid delivery. Thus, while the fluid is allowed to discharge through a portion of the openings, uniform fluid delivery across all of the openings is impaired.

Typically, to remove the air, a user primes the elongated tube using a syringe by injecting the fluid at a high flow rate and pressure into the open proximal end of the elongated tube while the elongated tube is disconnected from the fluid source. This action is intended to drive or push the air out through the plurality of openings. However, even if the priming of the elongated tube removes a large percentage of the air, the air may infiltrate in through the open proximal end when the user removes the syringe and makes the connection with the fluid source. In addition, assuming the priming removes most of the air, air can still be infiltrated from other upstream points, such as air traps in the tubing, valves, or the fluid source. Hence, even if the elongated tube is primed, not all of the air is removed or prevented from appearing later.

In addition to air blocking the flow of the fluid, kinking of the elongated tube of the catheter may also result in blocking the flow of the fluid, much like a garden hose when it becomes kinked. Prior art catheters have attempted to overcome the problems associated with air blockage and kinking by placing reinforcement members within the lumen. One such catheter is shown in U.S. Pat. No. 6,676,643 to Brushey. In Brushey, a coil is positioned in the lumen of the elongated tube. The coil abuts the inner surface of the elongated tube and includes spaced convolutions to allow the fluid to flow through the coil and out the plurality of openings. Both proximal and distal ends of the coil in Brushey are fixed to the inner surface. By abutting the coil against the inner surface of the elongated tube and attaching both the proximal and distal ends of the coil to the inner surface, the coil stiffens the catheter to provide kink resistance.

### BRIEF SUMMARY OF THE INVENTION AND ADVANTAGES

The present invention provides a catheter for uniformly delivering fluid to an anatomical site of a patient. The catheter comprises an elongated tube having an open proximal end and a closed distal end. The elongated tube includes a catheter wall having an inner surface with a lumen bound by the inner surface between the proximal and distal ends. A plurality of openings are defined in the catheter wall for discharging fluid from the lumen to the anatomical site of the patient. A coil is disposed within the lumen. The coil includes spaced convolutions in an unstretched state. A distal end of the coil is fixed to the distal end of the elongated tube and the coil extends unattached in the lumen along a length thereof to a proximal end. The coil is spaced from the inner surface of the catheter wall along the length to define an annular flow space therewith.

One advantage of this configuration is the ability to provide kink resistance to the catheter by way of the coil in the lumen, while at the same time providing an additional flow path to the plurality of openings, i.e., via the annular flow space. As a result, should air bubbles become trapped in the annular flow space, then the fluid can still travel through the coil and out between the spaced convolutions to the plurality of openings and vice versa. Furthermore, by fixing the coil at only one end, i.e., the distal end, with the remainder of the coil being unattached in the lumen, the coil provides adequate kink resistance while maintaining flexibility.

A method of manufacturing the catheter is also provided. The method includes first positioning the coil within the lumen of the elongated tube. Next, the distal end of the coil is positioned within a spacer tube and the spacer tube is inserted between the coil and the inner surface of the elongated tube to define the annular flow space. Once the spacer tube is positioned in the distal end of the elongated tube with the distal end of the coil, the coil is embedded in the spacer tube and the distal end of the elongated tube. This fixes the distal end of the coil, while allowing the remainder of the coil to move freely within the elongated tube.

### BRIEF DESCRIPTION OF THE SEVERAL VIEWS OF THE DRAWINGS

Advantages of the present invention will be readily appreciated as the same becomes better understood by reference to the following detailed description when considered in connection with the accompanying drawings wherein:

FIG. 1 is an exploded view of a catheter of the present invention;

FIG. 2 is an assembled view of the catheter of FIG. 1;

FIG. 3 is a cross-sectional view illustrating a connection between an adapter tube and an elongated tube of the present invention;

FIG. 4 is a broken elevational view illustrating continuous and infusion segments of the elongated tube;

FIG. 5 is an end view of the elongated tube shown in FIG. 4;

FIG. 6 is an elevational view of the infusion segment of the elongated tube;

FIG. 7 is a cross-sectional view of the infusion segment of the elongated tube taken along the line 7-7 in FIG. 6;

FIG. 8 is a cross-sectional view of the infusion segment of the elongated tube taken along the line 8-8 in FIG. 6;

FIG. 9 is a cross-sectional assembly view illustrating assembly of a coil and a spacer tube into the elongated tube of the present invention;

FIG. 10 is an exploded view of a tube set that can be used with the catheter of the present invention; and

FIG. 11 is an assembled view of the tube set of FIG. 10.

### DETAILED DESCRIPTION OF THE INVENTION

Referring to the Figures, wherein like numerals indicate like or corresponding parts throughout the several views, a catheter for delivering fluid to an anatomical site of a patient is generally shown at 10. It is to be appreciated that the fluid is preferably medication for pain management, but other fluids such as a saline solution, medication for other purposes, and the like could also be used.

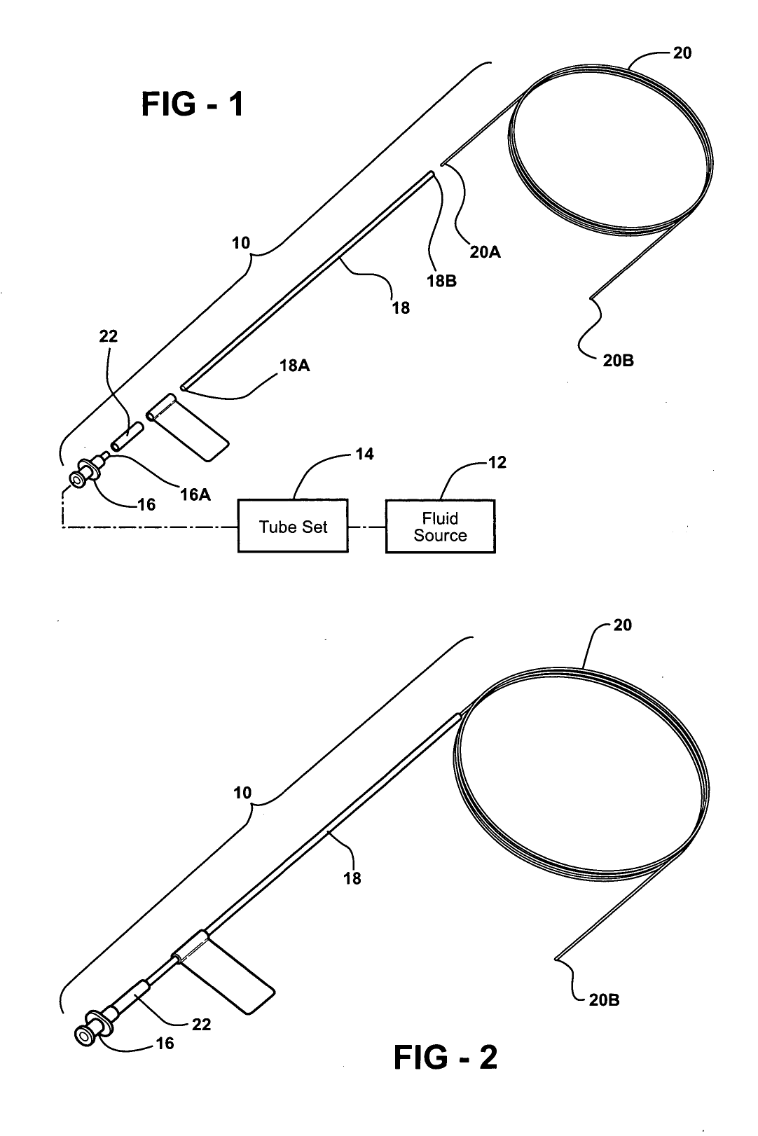

Referring to FIG. 1, the catheter 10 forms part of a fluid delivery system that includes a fluid source generally indicated at 12 to supply the fluid to be delivered to the anatomical site and a tube set generally indicated at 14 to convey the fluid from the fluid source 12 to the catheter 10. The fluid source 12 can include any device or manner to store and/or deliver the fluid to the anatomical site of the patient. Such devices may include, but are not limited to, pain medication pumps, intravenous bags, and the like.

The catheter 10 is in fluid communication with the fluid source 12 and the tube set 14 and conveys the fluid to the anatomical site. A fluid connection mechanism 16, such as a female luer connector 16, couples the catheter 10 with the tube set 14. The fluid connection mechanism 16 may also be a threaded fitting, snap fitting, or any type of fitting used by those skilled in the art to interlock the catheter 10 with the tube set 14. The catheter 10 may also be bonded to the tube set 14, welded to the tube set 14, or integrally formed therewith. It should be appreciated that the catheter 10 may also engage the fluid source 12 directly.

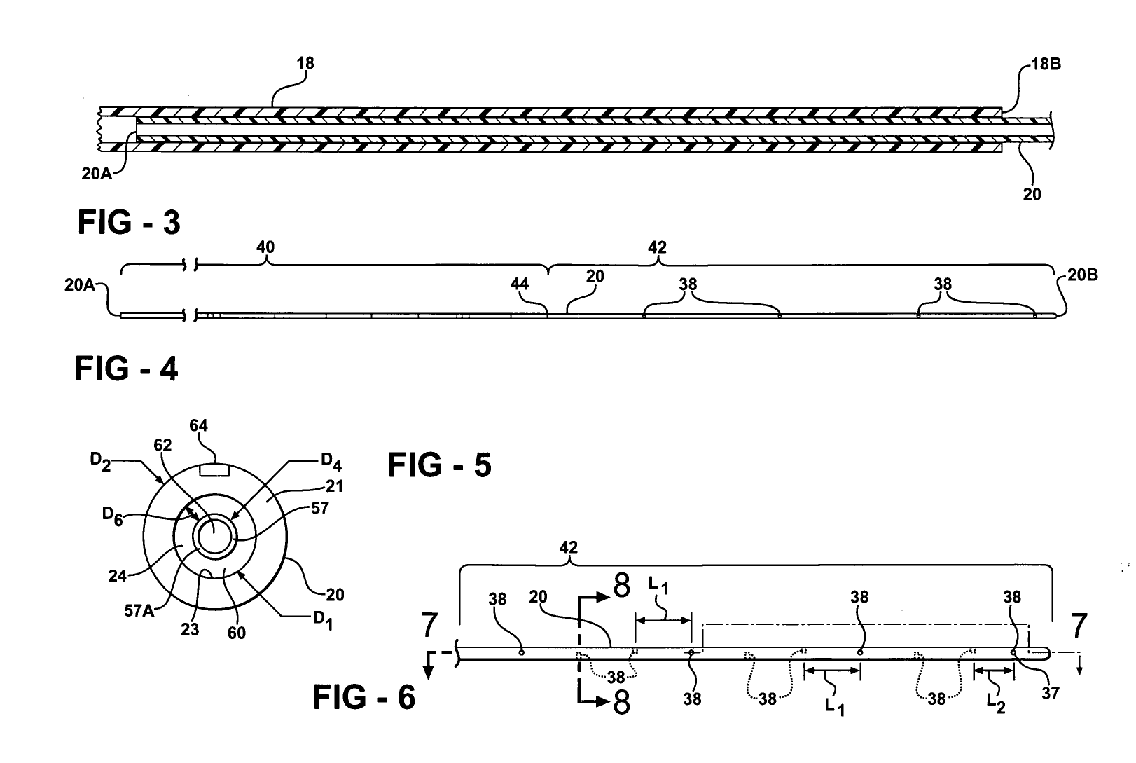

The catheter 10 includes an adapter tube 18 having proximal 18A and distal 18B ends and an elongated tube 20 having an open proximal end 20A partially protruding into said adapter tube 18 at the distal end 18B thereof. See FIG. 3. The open proximal end 20A is sealed inside the adapter tube 18 using a UV curable adhesive such as Dymax® 204 CTH-F. The elongated tube 20 extends from the open proximal end 20A inside the adapter tube 18 to a closed distal end 20B, as shown in FIG. 2.

An adapter bushing 22 extends between the connector 16 and the proximal end 18A of the adapter tube 18. More specifically, the adapter bushing 22 fits over a port 16A of the connector 16 and is fixed and sealed thereto, using cyclohexanone. Similarly, the proximal end 18A of the adapter tube 18 fits inside the adapter bushing 22 and is fixed and sealed thereto, also using cyclohexanone. The connector 16 could also be attached directly to the adapter tube 18 or the elongated tube 20.

Referring to FIG. 4, the elongated tube 20 is shown. The elongated tube 20 may be any length, but in one embodiment is about 36.0 inches in length. The elongated tube 20 includes a continuous segment 40 and an infusion segment 42. These segments merely define boundaries of the elongated tube 20 and do not indicate breaks or connection points in the elongated tube 20. The infusion segment 42 in one embodiment is 5.0 inches in length, but other lengths are possible for various uses of the catheter 10. The continuous segment 40 extends from the open proximal end 20A to an intermediate point 44 and includes a series of graduated marks to assist a user in determining the depth in which the elongated tube 20 has been inserted into the anatomical site. The infusion segment 42 extends from the intermediate point 44 to the closed distal end 20B and includes a plurality of openings 38 that discharge the fluid out from the elongated tube 20 to the anatomical site. The continuous segment 40 is devoid of openings, except for the open proximal end 20A.

The elongated tube 20 can be made from any number of materials including, but not limited to, any appropriate flexible, sterilizable, biocompatible polymer or plastic. Some typical materials include silicone, polyamides, clear polyether-block co-polyamides (PEBAX) and copolymers thereof, as well as polyurethanes, low-density polyethylene, high-density polyethylene, polypropylene, polystyrene, polycarbonate, polytetrafluoroethylene, tetrafluoroethylene, and fluorinated ethylene propylene.

Referring to FIG. 5, which illustrates an end view of the elongated tube 20 shown in FIG. 4, the elongated tube 20 includes a catheter wall 21 having an inner surface 23. A lumen 24 is bounded by the inner surface 23 between the open proximal end 20A and the closed distal end 20B of the elongated tube 20. The lumen 24 has a diameter D₁ that ranges from 0.025 to 0.031 inches. The elongated tube has an outer diameter D₂ that ranges from 0.047 to 0.049 inches. A reinforcing coil 57 is disposed in the lumen 24. The reinforcing coil 57 has an outer diameter D₄ that ranges from 0.014 to 0.017 inches. The outer diameter D₄ of the reinforcing coil 57 is smaller than the diameter D₁ of the lumen 24 to define an annular flow space 60 therebetween to improve fluid flow from the fluid source 12 to the anatomical site. The annular flow space 60 has a depth D₆ defined radially between the reinforcing coil 57 and the inner surface 23 that ranges from 0.004 to 0.01 inches. The depth D₆ of the annular flow space 60 is preferably greater than or equal to a diameter D₃ of each of the plurality of openings 38. See briefly FIG. 8.

Referring to FIG. 6, the openings 38 are axially spaced from adjacent openings 38 along the infusion segment 42 and the openings 38 are also circumferentially spaced about a central axis of the infusion segment 42 by one hundred twenty degrees from adjacent openings 38. Of course, other circumferential spacing of the openings 38 could also be employed. Hidden lines illustrate openings 38 normally not visible in this elevational view.

One of the plurality of openings 38 in the infusion segment 42 is further defined as a vent hole 37. The vent hole 37 is used to remove residual air that may otherwise be trapped in the lumen 24 of the elongated tube 20 during priming of the catheter 10. When the catheter 10 is being primed, using methods well known to those skilled in the art, air is forced out through the plurality of openings 38 and the vent hole 37. The plurality of openings 38 are equally spaced from one another along the elongated tube 20 by a first length L₁, preferably about 0.50 inches. The vent hole 37 is the distal most opening 38 and is axially spaced by a second length L₂. The second length L₂ is shorter than the first length L₁. The second length L₂ is preferably about 0.25 inches. This configuration allows the plurality of openings 38 to maintain uniform fluid delivery along the infusion segment 42. In alternative embodiments, the plurality of openings 38 are spaced at different lengths from axially adjacent openings and each of these differing lengths are greater than the second length L₂.

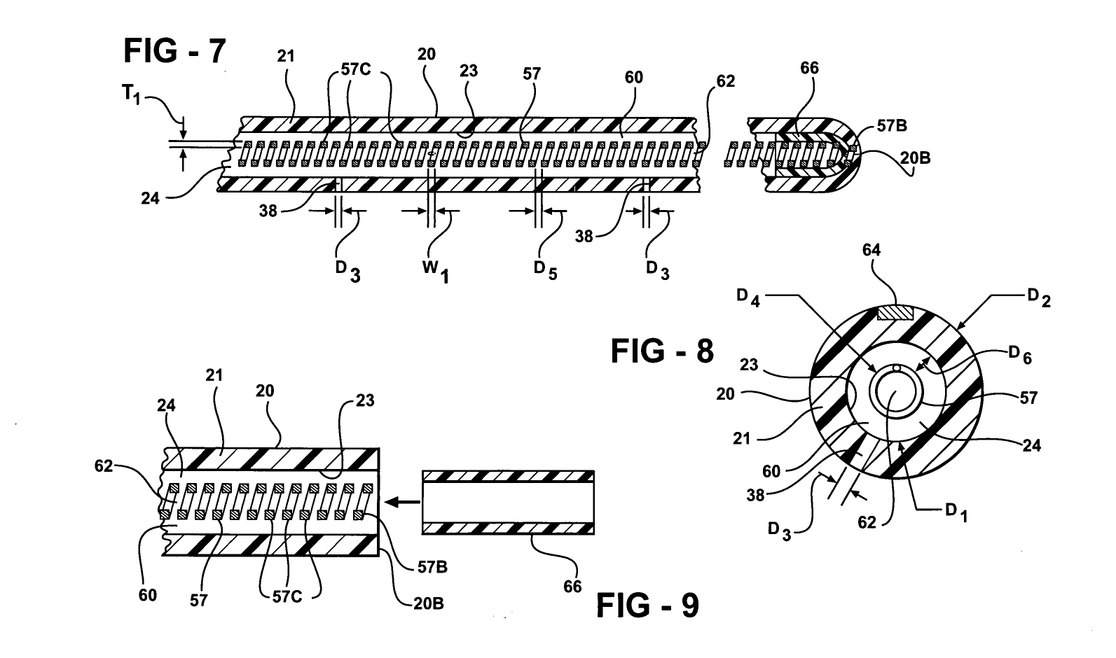

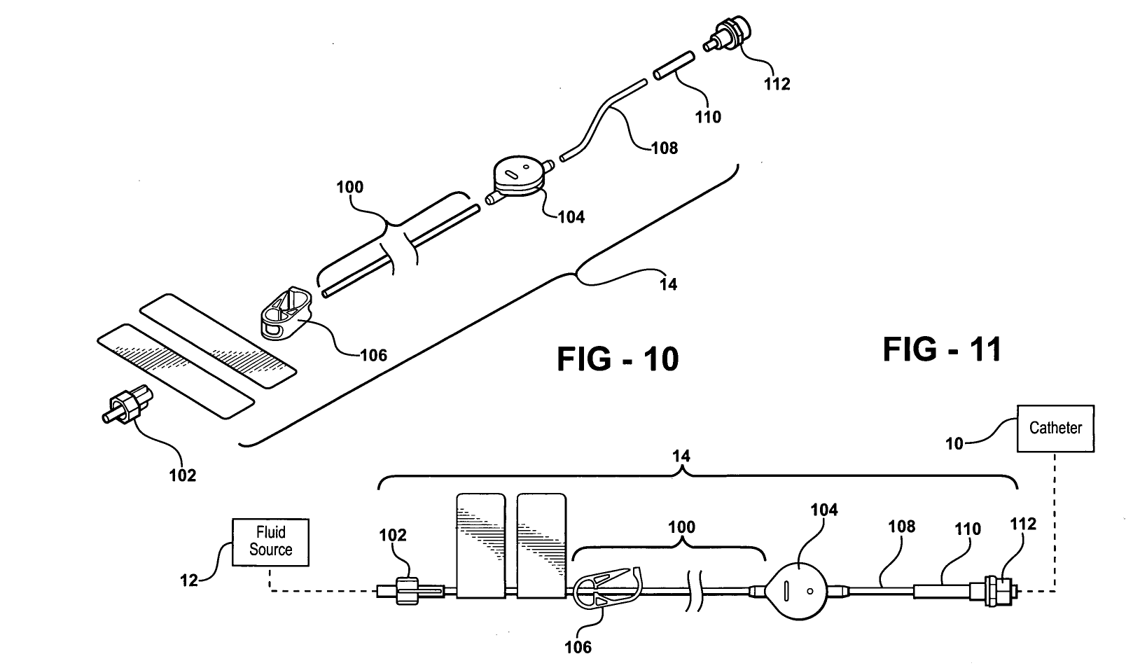

Referring to FIGS. 7 and 8, the diameter D₃ of each of the plurality of openings 38 ranges from 0.004 to 0.006 inches. In the Figures, the openings 38 assume a circular shape, but the openings 38 may take on a variety of shapes including elliptical, rectangular, triangular, or a combination of two or more different shapes. Should the openings 38 assume a non-circular shape, then the diameter D₃ of such openings would be the equivalent circular diameter based on the cross-sectional area of the openings. The openings 38 may be micro-drilled or laser-drilled in the catheter wall 21 or formed by other conventional methods.

Still referring to FIG. 7, the reinforcing coil 57 extends between a proximal end 57A (See FIG. 5) and a distal end 57B. The distal end 57B of the reinforcing coil 57 is embedded in the closed distal end 20B of the elongated tube 20. In particular, as will be appreciated by those skilled in the art, the closed distal end 20B of the elongated tube 20 is formed by radio frequency (RF) tipping techniques and the reinforcing coil 57 is embedded and fixed in a tip of the elongated tube 20 at the closed distal end 20B during this process. From the distal end 57B of the reinforcing coil 57, the reinforcing coil 57 extends proximally and unattached along a length thereof to the proximal end 57A of the reinforcing coil 57, which also remains unsupported and unattached within the lumen 24, i.e., freely floats within the lumen 24. The reinforcing coil 57 reinforces the elongated tube 20 and prevents kinking of the elongated tube 20 to ensure uniform fluid delivery. The reinforcing coil 57 has spaced convolutions 57C in an unstretched state to define an open coil. Here the convolutions 57C are spaced apart a distance D₅ that ranges from 0.004 to 0.005 inches, which approximates the diameter D₃ of each of the openings 38. At the same time, each of the convolutions 57C have a rectangular cross-section with a width W₁ that ranges from 0.003 to 0.005 inches and a thickness T₁ that ranges from 0.001 to 0.003 inches. The reinforcing coil 57 may be made from any appropriate sterilizable and biocompatible material, such as 304 stainless steel, titanium, nickel-titanium and plastic filament.

The reinforcing coil 57 is coaxially positioned within the lumen 24 at the closed distal end 20B of the elongated tube 20. This is accomplished by using a spacer tube 66 that is coaxially and snugly positioned in the lumen 24 at the distal end 20B of the elongated tube 20. The distal end 57B of the reinforcing coil 57 fits coaxially within the spacer tube 66 to define the annular flow space 60. The spacer tube 66 and the distal end 20B of the elongated tube 20 are tipped together with the distal end 57B of the reinforcing coil 57 positioned to embed the reinforcing coil 57 in the spacer tube 66 and the elongated tube 22. The spacer tube 66 is preferably colored to provide an indication to a user when the distal end 20B of the elongated tube 20 fully exits the anatomical site after use.

Referring to FIG. 9, a method of embedding the distal end 57B of the reinforcing coil 57 during manufacturing of the catheter 10 is shown. The method includes first positioning the reinforcing coil 57 within the lumen 24 of the elongated tube 20. Next, the spacer tube 66 is fitted over the distal end 57B of the reinforcing coil 57 and the spacer tube 66 is inserted in the distal end 20B of the elongated tube 20 between the reinforcing coil 57 and the inner surface 23 to define the annular flow space 60. Once the spacer tube 66 and reinforcing coil 57 are in position in the distal end 20B of the elongated tube 20, the spacer tube 66 and the elongated tube 20 are tipped together to fix and embed the reinforcing coil 57 therein.

The fluid moves through and about the reinforcing coil 57, as well as along the annular flow space 60 to reach the openings 38. The reinforcing coil 57 defines an inner lumen 62 along its length such that the fluid can travel either into the inner lumen 62 and between the spaced convolutions 57C of the reinforcing coil 57 into the annular flow space 60 of the elongated tube 20 to feed the plurality of openings 38, or the fluid can travel directly through the annular flow space 60 to the plurality of openings 38. By having multiple flow paths, blockages typically caused by air bubbles in the catheter 10 are reduced. For instance, should an air bubble infiltrate the lumen 24, the air bubble will likely be trapped, due to surface tension, between the reinforcing coil 57 and the inner surface 23 of the catheter wall 21. With the configuration set forth herein, fluid can still travel down the elongated tube 20 by traveling through the inner lumen 62 of the reinforcing coil 57, thus allowing fluid to continue past the air bubble and exit through the openings 38.

Referring to FIGS. 5 and 8, a radiopaque stripe 64, such as those formed from tungsten, may be extruded in the catheter wall 21.

An example of a tube set 14 that can be used to interconnect the fluid source 12 with the catheter 10 is shown in FIGS. 11 and 12. In this instance, the tube set 14 includes a first tube 100 interconnecting an extended luer connector 102 for connecting to the fluid source 12 and an inline filter 104 for filtering microbes and air from the fluid. An on/off clamp 106 can be positioned about the tube 100 to manually control fluid flow. A second tube 108 and an adapter bushing 110 interconnect the inline filter 104 with a male luer connector 112. Connections between these components can be made using cyclohexanone, or other adhesive, as is well known to those skilled in the art. The extended luer connector 102 can be connected to the fluid source 12 and the female luer connector 16 of the catheter 10 can be attached to the male luer connector 112 to complete the fluid delivery system.

Obviously, many modifications and variations of the present invention are possible in light of the above teachings. The invention may be practiced otherwise than as specifically described within the scope of the appended claims.

What is claimed is:

1. A catheter for providing uniform fluid delivery to an anatomical site of a patient, comprising:
an elongated tube having an open proximal end and a closed distal end and including a catheter wall having an inner surface with a lumen bound by said inner surface between said proximal and distal ends;
a plurality of openings defined in said catheter wall for discharging fluid from said lumen to the anatomical site of the patient; and
a coil disposed within said lumen and including spaced convolutions in an unstretched state, said coil including a distal end fixed to said distal end of said elongated tube and extending unattached in said lumen along a length thereof to a proximal end with said coil being spaced from said inner surface of said catheter wall along said length to define an annular flow space therewith.

2. A catheter as set forth in claim 1 wherein said coil defines an inner lumen along said length such that the fluid can travel into said inner lumen and between said spaced convolutions of said coil into said annular flow space of said elongated tube to feed said plurality of openings which discharge the fluid to the anatomical site.

3. A catheter as set forth in claim 2 wherein each of said plurality of openings has a diameter and said annular flow space has a depth defined between said coil and said inner surface of said lumen that is greater than or equal to said diameter of each of said plurality of openings.

4. A catheter as set forth in claim 3 wherein each of said spaced convolutions are axially spaced a distance from adjacent convolutions with said distance being approximately the same as said diameter of each of said plurality of openings.

5. A catheter as set forth in claim 3 wherein said diameter of each of said plurality of openings ranges from 0.004 to 0.006 inches, said depth of said annular flow space ranges from 0.004 to 0.01 inches.

6. A catheter as set forth in claim 5 wherein each of said plurality of openings is micro drilled in said catheter wall.

7. A catheter as set forth in claim 1 wherein said spaced convolutions are axially spaced at a range of 0.004 to 0.005 inches apart.

8. A catheter as set forth in claim 7 wherein each of said spaced convolutions has a width that ranges from 0.003 to 0.005 inches, a thickness that ranges from 0.001 to 0.003 inches, and said coil has an outer diameter that ranges from 0.014 to 0.017 inches.

9. A catheter as set forth in claim 1 wherein said lumen of said elongated tube has a diameter that ranges from 0.025 to 0.031 inches.

10. A catheter as set forth in claim 9 wherein said elongated tube has an outer diameter that ranges from 0.047 to 0.049 inches.

11. A catheter as set forth in claim 2 including a spacer tube coaxially and snugly positioned in said lumen at said distal end of said elongated tube wherein said distal end of said coil fits coaxially within said spacer tube to define said annular flow space and said spacer tube and said elongated tube are tipped together with said coil using radio frequency to embed said coil in said distal end of said elongated tube.

12. A catheter as set forth in claim 11 wherein said spacer tube is colored to provide an indication to a user when said distal end of said elongated tube fully exits the anatomical site after use.

13. A catheter as set forth in claim 1 wherein said distal end of said elongated tube includes a tip and said distal end of said coil is embedded in said distal end of said elongated tube at said tip.

14. A catheter as set forth in claim 1 including a radiopaque stripe extruded in said catheter wall.

15. A catheter as set forth in claim 1 wherein said elongated tube is formed from sterilizable plastic.

16. A catheter as set forth in claim 15 wherein said sterilizable plastic is selected from the group consisting of polyurethanes, low density polyethylene, high density polyethylene, polypropylene, polystyrene, polycarbonate, polytetrafluoroethylene, tetrafluoroethylene, fluorinated ethylene propylene and polyamides.

17. A catheter as set forth in claim 15 wherein said sterilizable plastic comprises polyether-block co-polyamides and copolymers thereof.

18. A catheter as set forth in claim 1 wherein said coil is made from a material selected from the group consisting of stainless steel, titanium, nickel-titanium and plastic filament.

19. A method of manufacturing a catheter having an elongated tube defining a lumen and a plurality of openings therein to convey fluid to an anatomical site of a patient with a coil disposed within the lumen to provide uniform fluid delivery and kink resistance, the coil being spaced from an inner surface of the elongated tube by a spacer tube to define an annular flow space between the inner surface and the coil, said method comprising the steps of:
positioning the coil within the lumen of the elongated tube;
positioning the spacer tube over a distal end of the coil and inserting the spacer tube between the coil and the inner surface of the elongated tube to define the annular flow space; and
embedding the distal end of the coil in the distal end of the elongated tube once the coil is positioned within the lumen, once the spacer tube is positioned over the distal end of the coil, and once the spacer tube is inserted between the coil and the inner surface of the elongated tube at the distal end of the elongated tube.

20. A method as set forth in claim 19 wherein embedding the distal end of the coil in the distal end of the elongated tube includes tipping the spacer tube and the elongated tube together using radio frequency to embed the coil within the tipped tubes.
